(19) United States
(12) Patent Application Publication
(10) Pub. No.: US 2008/0262475 A1
(43) Pub. Date: Oct. 23, 2008

(54) ADAPTER FOR AN INTRODUCER

(75) Inventor: Fredrik PREINITZ, Uppsala (SE)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 11/738,023

(22) Filed: Apr. 20, 2007

(51) Int. Cl. A61M 39/10

(52) U.S. Cl. 604/533

(57) ABSTRACT

An adapter is provided for use in connecting to a percutaneous introducer of arbitrary dimensions, where the adapter comprises a tapered threaded section (4). The tapered threaded section is screwed (and/or inserted) into the introducer and will thereby tightly grip the inner walls of the introducer. In one embodiment the adapter is provided as the distal section of a new introducer (3), required for a subsequent medical procedure. In another embodiment, the adapter is provided as an integral part of a medical device (16), such as an insertion tool for a closure device. In a further embodiment the distal end (30) of the tapered threaded section (24) is extended beyond the distal end of the introducer.

### FIELD OF THE INVENTION

The present invention relates generally to an adapter for an introducer which through a percutaneous puncture is used to obtain access to blood vessels, cavities, organs or other bodily tissues in order to perform a medical operation, and more particularly to an adapter being provided with a tapered threaded end, which is screwed or inserted into an introducer already in place at the puncture site.

### BACKGROUND OF THE INVENTION

An introducer is an elongated tubular member, which in the field of medical surgery is used to gain access to a particular site within a patient's body. It should be noted that the term introducer is, unless otherwise indicated, used herein as indicative of an introducer sheath which comprises at least one proximal entry port. Depending on the medical procedure, the design of an introducer can vary. For example, the proximal end can comprise an attachment means for a medical instrument. Additionally, an entry port can comprise sealing material to prevent leakage of e.g. bodily fluids. Furthermore, an introducer can comprise several entry ports.

Usually the medical operation wherein an introducer is used commences with a puncture operation in which a hollow needle is introduced at a point on a patient's skin, and is then advanced through tissues beneath the skin to the position in the organ of interest, e.g. a blood vessel. A guide wire is then introduced through the needle, whereupon the needle is removed, and an introducer together with a dilator is advanced over the guide wire. After removal of the guide wire and the dilator, access to the organ has now been obtained through the lumen of the tubular introducer.

The procedure described above can be used to gain access to a blood vessel for performing different types of intravascular operations. When the operation in question is completed, the puncture hole in the blood vessel wall can be sealed by a closure device, wherein an inner member of said closure device is positioned at an inner surface of the vessel wall by means of an insertion device and which is held in place by a suture or filament. The suture or filament extends from the closure device, through tissue overlaying the vessel, and out of the skin surface. An example of this technique is disclosed in U.S. Pat. No. 4,744,364.

In many cases several different medical procedures need to be performed on a patient using the same access site, such as a percutaneous blood vessel puncture. Some examples of different procedures using such a puncture are insertion of and measurements using a sensor guide wire, placing a stent, performing angioplasty and, in most cases, sealing the puncture, as described above. Especially in the case of sealing the puncture, which follows after one or several different medical procedures, a need arises to switch introducer sheaths, due to specific requirements on the access path of the closure device.

### SUMMARY OF THE INVENTION

As mentioned above, introducers are of many different dimensions and/or designs. Furthermore, the introducer can frequently be an integrated part of a dedicated instrument. Although the introducers in prior art serve their intended purposes, changing the introducers increases the risk of causing infection, excessive bleeding due to ruptures of blood vessels, and discomfort or pain to the patient. Prior art shows several examples of adapters to be able to connect to an introducer. Two such examples are described in EP1305076 and U.S. Pat. No. 5,147,336. However, these adapters require a specific shape or dimension of a matching mating member on the introducer. Therefore there is a need for a way to use an introducer which is already in place in e.g. a blood vessel for a subsequent medical procedure, irrespective of the shape and dimensions of the introducer.

An object of the present invention is to provide an adapter which can be inserted into a pre-placed introducer of arbitrary dimensions, eliminating the need to replace the introducer. A further object of some embodiments of the invention is to provide an adapter which can be used to connect any pre-placed introducer with any new introducer, without having to remove the first introducer. Yet an additional object of some embodiments of the invention is to provide an adapter comprising an insertion tool for a closure device.

According to one embodiment of the present invention a universal introducer adapter comprises a tapered threaded section which is attached to the pre-placed introducer by screwing the adapter directly into the introducer sheath. The tapered shape allows the attachment to introducers with a wide range of inner dimensions. The threads provide means to firmly attach the adapter and seal the passageway from leakage of e.g. blood or other bodily fluids. Using the adapter eliminates the need for replacement of the introducer when switching medical procedures requiring introducer sheaths with different dimensions and/or different configurations. According to one embodiment the adapter with a tapered threaded end is the distal end of a new introducer, needed for a subsequent medical procedure. According to another embodiment the adapter is an integrated part of a medical device, such as an insertion tool for a closure device. In a further embodiment, the distal end of the tapered threaded section comprises an extended tubular section, which reaches beyond the distal end of the pre-placed introducer.

### BRIEF DESCRIPTION OF THE DRAWINGS

FIGS. 1a and 1b illustrate the present invention according to a first embodiment.

FIG. 2 illustrates the present invention according to a second embodiment.

FIG. 3 illustrates the present invention according to a third embodiment.

FIG. 4 illustrates the present invention according to a fourth embodiment.

FIG. 5 illustrates the present invention according to a fifth embodiment.

FIG. 6 illustrates the present invention according to a sixth embodiment.

### DETAILED DESCRIPTION OF PREFERRED EMBODIMENTS

The present invention will be described in the context of a percutaneous puncture made to gain access to a blood vessel. However, it is within the scope of the present invention to use the present invention in other procedures, such as obtaining access to the abdominal cavity or a specific organ through an introducer.

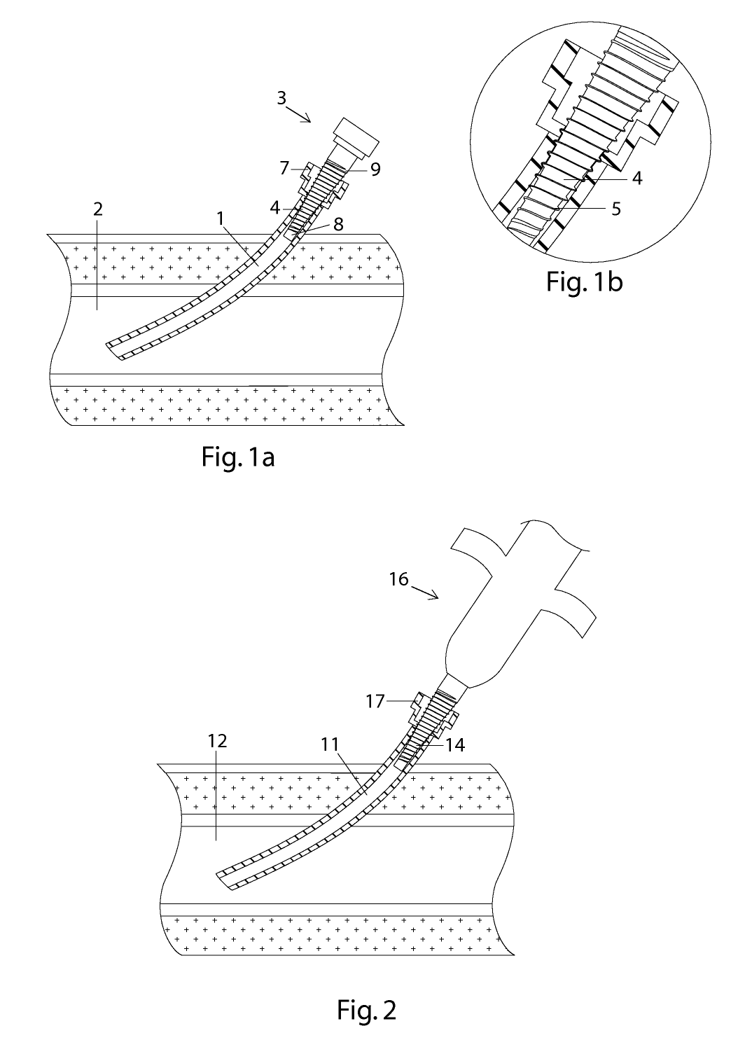

A first embodiment of the present invention is illustrated in FIGS. 1a and 1b. A pre-placed introducer 1 is present in a percutaneous puncture into e.g. a blood vessel 2. This introducer can have been used for a variety of purposes, such as insertion of a guide wire, the drawing of a blood sample or infusion of a solution into the blood stream. Depending on the purpose and the retailer of said introducer, it can have a variety of dimensions and shapes. The introducer 1 in FIG. 1a is a simple schematic of such an introducer. Normally the inner dimension of an introducer is between 3 French and 10 French (approximately 1.0 mm and 3.3 mm, respectively). In this embodiment, the present invention comprises a new introducer 3, necessary for a subsequent procedure, comprising a tapered distal section 4 provided with a plurality of external threads 5. This is illustrated in FIG. 1a with a magnification of the tapered section in FIG. 1b. Introducer 3 is provided with an inner lumen extending from the proximal end to the distal end, providing access to the inner lumen of the pre-placed introducer 1, and thereby also to the lumen of blood vessel 2, through introducer 3.

The following is described in reference to FIGS. 1a and 1b, but is also applicable to the embodiments described in FIGS. 2-4. The distal tapered section 4 is attached by screwing introducer 3 into the pre-placed introducer 1 such that at least one full turn or rotation of the threads grips the inner wall of the pre-placed introducer 1 (see magnification in FIG. 1b). This produces a secure tight seal between the pre-placed introducer and the present invention, in order to minimize loss of bodily fluids such as blood. Due to the tapered shape of the tapered section 4, the present invention can attach to a variety of different inner dimensions of the pre-placed introducer 1. Furthermore, it is also within the scope of the present invention that the threads attach to a more proximal part of the pre-placed introducer, such as a softer seal in a hub 7 of the introducer 1, often provided to prevent blood loss when accessing a blood vessel.

As described earlier herein, the term introducer comprises an introducer sheath and, if present, an integrated cap, valve or hub. The following is described in reference to FIGS. 1a and 1b, but is also applicable to the embodiments described in FIGS. 2-6. To be able to attach to the inner walls of a pre-placed introducer, the maximum external cross-sectional dimension of the narrowest part 8 of the tapered section 4 is smaller than a distal inner dimension of the pre-placed introducer, and the external cross-sectional dimension of a widest part 9 of the tapered section 4 exceeds that of a proximal inner dimension of the pre-placed introducer. Thereby the present invention, when screwed or inserted into a pre-placed introducer, will grip and seal tight along an arbitrary section of the threads of the tapered section and the inside of the pre-placed introducer. This is illustrated in Figures.

It should be noted that it is also within the scope of the present invention that the tapered section, as mentioned above, grips and seals tight against a section inside the cap, valve or hub of the pre-placed introducer. In the latter case, this can provide the advantage of not decreasing the diameter of the available inner lumen of the introducer when attached to the adapter. Furthermore, in this context, it should also be noted that it is within the scope of the present invention that the abovementioned inner diameters of the pre-placed introducer allow for resilience by employing a soft or pliable material. A non-limiting example of such a situation is when a soft sealing material, such as silicone or polyurethane, is provided as a gasket in an introducer's hub, which is displaced by insertion of the adapter of the present invention.

In a second embodiment, illustrated in FIG. 2, a tapered distal section 14 is an integral part of a medical device 16, such as one used to insert an instrument or perform closure of the percutaneous puncture. Also in this embodiment, the tapered section is provided with a plurality of external threads (not labeled in FIG. 2, refer to FIG. 1b). FIG. 2 also illustrates a hub 17.

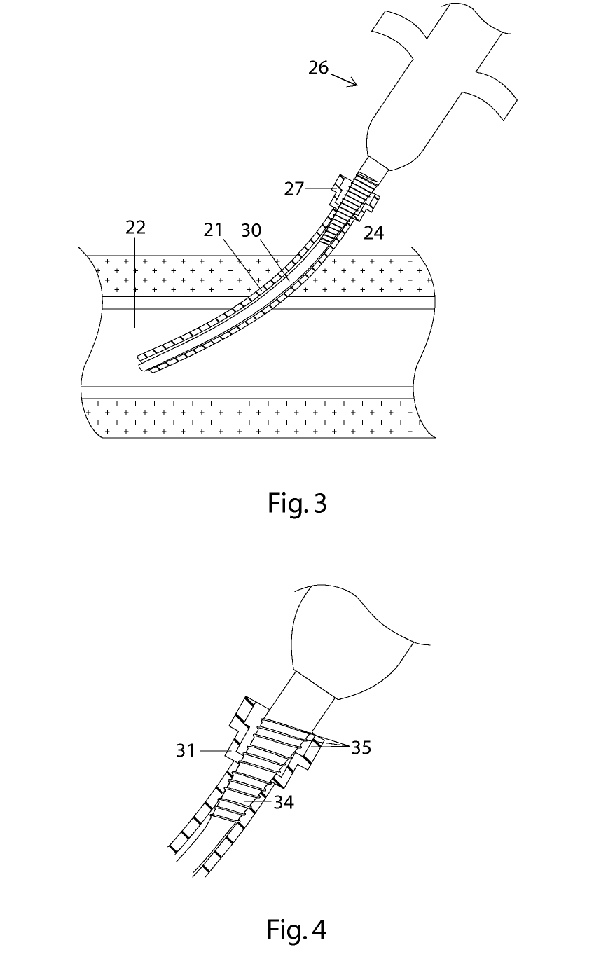

In a further embodiment, a distal end 30 of the present invention is extended such that the tip reaches beyond the distal end of a pre-placed introducer 21. This is illustrated in FIG. 3. When e.g. performing a closure procedure of the percutaneous puncture, it is advantageous if the dimensions of the entire access route of sealing discs are compatible with the medical device used. By extending the distal end, the delivery of e.g. a sealing disc is not compromised due to a change in size and/or shape of the delivery channel while transporting the disc from the medical device into the blood vessel. It should be noted that in this embodiment the extended section 30, distal to the tapered section 24, is preferably made of a material that is soft enough to follow the shape of the pre-placed introducer, without inhibiting the manipulation of the introducer.

Similar to the embodiments in FIGS. 1a, 1b and 2, the third embodiment in FIG. 3 is provided with an inner lumen extending from the distal end, which, when the device is placed in a blood vessel 22, provides access to the lumen of said blood vessel 22, up into the interior of a medical device 26. FIG. 3 also illustrates a hub 27.

In FIGS. 1-3, the threads are illustrated as approximately 18 rotations or turns around the tapered section 4, 14, 24. As used herein, the term “threads” encompasses the type of arrangement shown in FIG. 1b. That is, FIG. 1b shows “threads” even though there is only one continuous projection forming item 5 in FIG. 1b. It should be noted that it is within the scope of the present invention that the provided number of rotations of the threads around the tapered section is more than one full turn, to provide good sealing properties. However, to achieve a wide range of use for the adapter, it can be provided with up to 100 rotations or more. In addition, the threads can be provided as several parallel threads. This is illustrated in FIG. 4, where three parallel threads 35 extend in a spiral or coil along the tapered section 34. Due to the steep turn in such an arrangement, the tapered section 34 obtains faster gripping action (i.e. necessitating less effort) when screwing the adapter into the pre-placed introducer. It should be noted that FIG. 4 illustrates a non-limiting example, and that the number of parallel threads 35 can also be two, four or more.

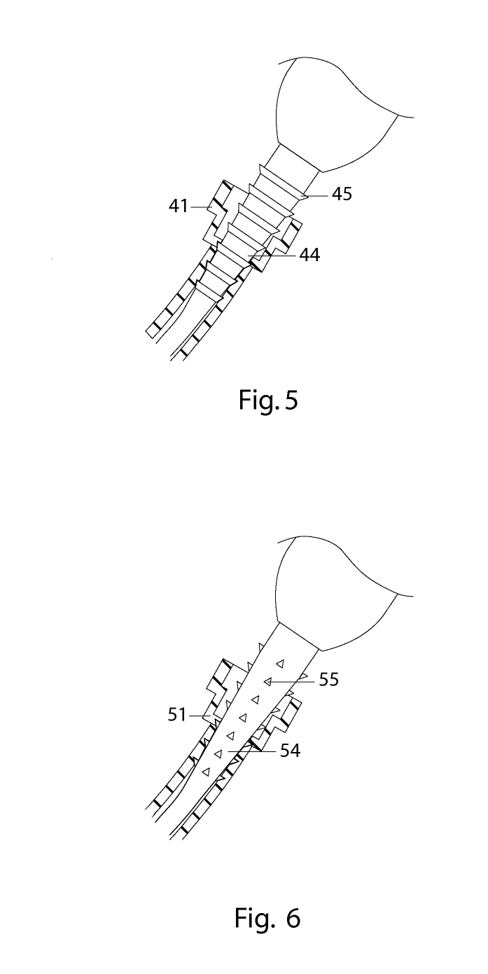

Yet another embodiment of the present invention is illustrated in FIG. 5. Here a tapered section 44 is provided with threads or ridges 45 that are perpendicular to the central axis of the tapered section. The ridges 45 thereby form circumferential protrusions. Furthermore, the outer edges of the ridges are preferably angled, such that when the adapter is inserted or screwed into the pre-placed introducer, the ridges will allow easy insertion, while providing resistance when pulling back on the adapter. In a further embodiment, illustrated in FIG. 6, the ridges are replaced by several barbs or hooks 55, which are randomly or systematically positioned on the surface of the tapered section 54. These act as fasteners when the adapter is pulled back, such that the adapter is fitted within the introducer.

The material of the tapered section 4,14, 24, 34, if provided with threads, should preferably be harder than that of the walls of the pre-placed introducer 1, 11, 21, 31 such that the threads will seal tightly against the inner wall, by slightly deforming the inner wall (see FIGS. 1-4). Similarly, the tapered section 44, 54, if provided with ridges or barbs, as illustrated in FIGS. 5-6, can be of a harder material such that the ridges or barbs will penetrate or displace the walls of the pre-placed introducer 41, 51. Non-limiting examples of such materials are stainless steal, other metals, polycarbonate, polysulfonate, polyetheresterketone, other polymers, or carbon fiber composites. However, it should be noted that e.g. in the embodiment illustrated in FIG. 5, the threads or ridges 45 can comprise a softer material and the tapered section 44 a harder material, which also provides tight sealing action by the ridges against the inner wall of the pre-placed introducer. Similarly, in the embodiment in FIG. 6, the barbs 55 can be made of a softer material, providing resistance when retracting the adapter.

Although the present invention has been described with reference to specific embodiments it will be apparent to those skilled in the art that many variations and modifications can be performed within the scope of the invention as described in the specification and defined with reference to the claims below. For example, the adapter can be an integral part of an insertion tool for a closure device comprising an inner sealing disc and an outer sealing disc. Furthermore, the adapter can be an integral part of an insertion tool for a closure device comprising an inner anchor member and an outer sealing member, e.g. a collagen plug. In addition, the adapter can be provided as a separate member to be used together with insertion tools such as the two abovementioned tools. It should be noted that the term “gripping means” as used herein comprises threads, ridges, hooks, barbs or other protruding members that can act as fasteners to firmly hold the adapter positioned within an introducer. The embodiments described above can also be used in conjunction with the techniques and devices described in U.S. Pat. No. 7,073,509, U.S. Pat. No. 7,094,209 and U.S. Patent Application Publication No. 2004/0093025. All three of these documents are assigned to the assignee of this patent specification and are incorporated herein by reference in their entireties for such techniques and devices.

1. An adapter for use in connecting to a first percutaneous introducer wherein said adapter comprises a tapered distal section provided with gripping means.

2. An adapter according to claim 1, wherein said gripping means comprises threads.

3. An adapter according to claim 1, wherein said gripping means are chosen from the group comprising ridges, circumferential protrusions, hooks and barbs.

4. An adapter according to claim 1, wherein said tapered distal section is provided with dimensions such that a maximum external cross-sectional dimension of a narrowest part of the tapered distal section is smaller than a first inner dimension of the first percutaneous introducer, and an external cross-sectional dimension of a widest part of the tapered distal section exceeds that of a second inner dimension of the first percutaneous introducer.

5. An adapter according to claim 4, wherein said first inner dimension is at a position located more distal than a position of the second inner dimension.

6. An adapter according to claim 2, wherein said threads comprise two or more parallel threads.

7. An adapter according to claim 1, wherein said tapered distal section is an integrated part of a medical device.

8. An adapter according to claim 7, wherein said medical device comprises an insertion tool for a closure device.

9. An adapter according to claim 1, wherein said tapered distal section is an integrated part of a second introducer.

10. An adapter according to claim 1, wherein said tapered distal section is extended at a distal end such that a tip of the tapered distal section extends beyond a distal tip of said first percutaneous introducer when attached.

11. An adapter according to claim 1, wherein said tapered distal section is made of stainless steal, polycarbonate, polysulfonate, polyetheresterketone, or carbon fiber composites.
